(19) United States
(12) Patent Application Publication
(10) Pub. No.: US 2015/0150462 A1
(43) Pub. Date: Jun. 4, 2015

(54) SYSTEM AND METHOD FOR LOW COST PHOTOACOUSTIC IMAGING

(71) Applicant: General Electric Company, Schenectady, NY (US)

(72) Inventor: Mengli Wang, Niskayuna, NY (US)

(73) Assignee: General Electric Company, Schenectady, NY (US)

(21) Appl. No.: 14/094,814

(22) Filed: Dec. 3, 2013

(51) Int. Cl. A61B 5/00

(52) U.S. Cl. CPC A61B 5/0095; A61B 5/742; A61B 5/748

(57) ABSTRACT

A photoacoustic transducer assembly for imaging a subject of interest is presented. Furthermore, the photoacoustic transducer assembly includes a substrate. In addition, the photoacoustic transducer assembly includes a first plurality of source elements disposed on one or more sides of the substrate, wherein the first plurality of source elements is arranged along a periphery of the one or more sides of the substrate and configured to irradiate a region of interest in the subject of interest. Moreover, the photoacoustic transducer assembly also includes a plurality of detector elements disposed on the one or more sides of the substrate, wherein the plurality of detector elements is surrounded by the first plurality of source elements and configured to detect one or more signals generated by the region of interest in response to the irradiation.

### BACKGROUND

Embodiments of the present specification relate to imaging, and more particularly to low cost photoacoustic imaging.

Modern healthcare facilities often employ non-invasive imaging systems for identifying, diagnosing, and treating physical conditions. Medical imaging encompasses different non-invasive techniques used to image and visualize the internal structures and/or functional behavior (such as chemical or metabolic activity) of organs and/or tissues within a patient. Currently, a number of modalities of medical diagnostic and imaging systems exist, each typically operating on different physical principles to generate different types of images and information. These modalities include ultrasound systems, computed tomography (CT) systems, X-ray systems (including both conventional and digital or digitized imaging systems), positron emission tomography (PET) systems, single photon emission computed tomography (SPECT) systems, and magnetic resonance (MR) imaging systems.

In the recent years, photoacoustic imaging has shown potential as a viable imaging modality. As will be appreciated, photoacoustic imaging is based on the photoacoustic effect. In particular, photoacoustic imaging calls for the use of non-ionizing laser pulses as a source of irradiation. However, if radio-frequency (RF) pulses are used as the source of irradiation, the technique is referred to as thermoacoustic imaging. In photoacoustic imaging, a region of interest is irradiated with the non-ionizing laser pulses. The irradiated region of interest will absorb at least a portion of the delivered energy. The absorbed heat is converted into heat, thereby leading to thermoelastic expansion of the region of interest. As a consequence of this expansion ultrasonic waves are generated. The ultrasonic waves so generated are then detected by ultrasonic transducers. The photoacoustic imaging system uses these detected ultrasonic waves to form one or more images corresponding to the region of interest. Currently, photoacoustic imaging is being used in vivo for tumor angiogenesis monitoring, blood oxygenation mapping, functional brain imaging, and skin melanoma detection.

Presently available photoacoustic imaging systems entail use of a laser as the source of irradiation. For example, an yttrium aluminum garnet (YAG) laser is used as the source of irradiation in some currently available photoacoustic imaging systems. However, use of the YAG laser in the photoacoustic imaging system adds to the footprint of the photoacoustic imaging system. The increased footprint of the photoacoustic imaging system impedes portability of the system. Additionally, use of the YAG laser results in substantial increase in the cost of the photoacoustic imaging system. Moreover, the YAG laser based photoacoustic imaging system provides a single point source of illumination or irradiation and operates at a very low repetition rate, thereby resulting in increased scan time for scanning the region of interest. Certain other techniques have attempted to use a single laser diode or a fiber laser as the source of irradiation. However, use of the single laser source limits a laser beam profile and consequently a quality of the photoacoustic imaging system.

### BRIEF DESCRIPTION

In accordance with aspects of the present specification, a photoacoustic transducer assembly for imaging a subject of interest is presented. Furthermore, the photoacoustic transducer assembly includes a substrate. In addition, the photoacoustic transducer assembly includes a first plurality of source elements disposed on one or more sides of the substrate, where the first plurality of source elements is arranged along a periphery of the one or more sides of the substrate and configured to irradiate a region of interest in the subject of interest. Moreover, the photoacoustic transducer assembly also includes a plurality of detector elements disposed on the one or more sides of the substrate, where the plurality of detector elements is surrounded by the first plurality of source elements and configured to detect one or more signals generated by the region of interest in response to the irradiation.

In accordance with another aspect of the present specification, a photoacoustic imaging system for imaging a subject of interest is presented. The system includes an image data acquisition device configured to irradiate a region of interest in the subject of interest, where the image data acquisition device includes a photoacoustic transducer assembly, and where the photoacoustic transducer assembly includes a substrate, a first plurality of source elements disposed on one or more sides of the substrate, where the first plurality of source elements is arranged along a periphery of the one or more sides of the substrate and configured to irradiate the region of interest in the subject of interest, and a plurality of detector elements disposed on the one or more sides of the substrate, where the plurality of detector elements is surrounded by the first plurality of source elements and configured to detect one or more signals generated by the region of interest in response to the irradiation. Furthermore, the system includes an imaging device operatively coupled to the image data acquisition device and including an acquisition subsystem configured to acquire image data corresponding to the subject of interest from the image data acquisition device, and a processing subsystem in operative association with the acquisition subsystem and configured to process the acquired image data to generate one or more images corresponding to the region of interest in the subject of interest.

In accordance with yet another aspect of the present specification, a method for imaging a subject of interest is presented. The method includes irradiating a region of interest in the subject of interest using a photoacoustic transducer assembly, where the photoacoustic transducer assembly includes a substrate, a first plurality of source elements disposed on one or more sides of the substrate, where the first plurality of source elements is arranged along a periphery of the one or more sides of the substrate and configured to irradiate the region of interest in the subject of interest, and a plurality of detector elements disposed on the one or more sides of the substrate, where the plurality of detector elements is surrounded by the first plurality of source elements and configured to detect one or more signals generated by the region of interest in response to the irradiation. Additionally, the method includes detecting one or more signals generated by the region of interest in response to the irradiation. The method also includes generating one or more images using the detected one or more signals.

### DRAWINGS

These and other features, aspects, and advantages of the present specification will become better understood when the following detailed description is read with reference to the accompanying drawings in which like characters represent like parts throughout the drawings, wherein:

FIG. 1 is a diagrammatical illustration of an exemplary photoacoustic imaging system, in accordance with aspects of the present specification;

FIG. 2 is a diagrammatical representation of an exemplary photoacoustic transducer assembly for use in the system of FIG. 1, in accordance with aspects of the present specification;

FIG. 3 is a diagrammatical representation of another exemplary photoacoustic transducer assembly for use in the system of FIG. 1, in accordance with aspects of the present specification;

FIG. 4 is a diagrammatical representation of an exemplary method of making the photoacoustic transducer assembly of FIG. 2, in accordance with aspects of the present specification;

FIG. 5 is a flow chart depicting an exemplary method of imaging, in accordance with aspects of the present specification; and

FIG. 6 is a diagrammatical representation of an ultrasound imaging system for use in the system of FIG. 1, in accordance with aspects of the present specification.

### DETAILED DESCRIPTION

Systems and methods for enhanced photoacoustic imaging are presented. In particular, the photoacoustic imaging system presented herein provides a low cost, handheld, portable imaging system that enhances clinical workflow.

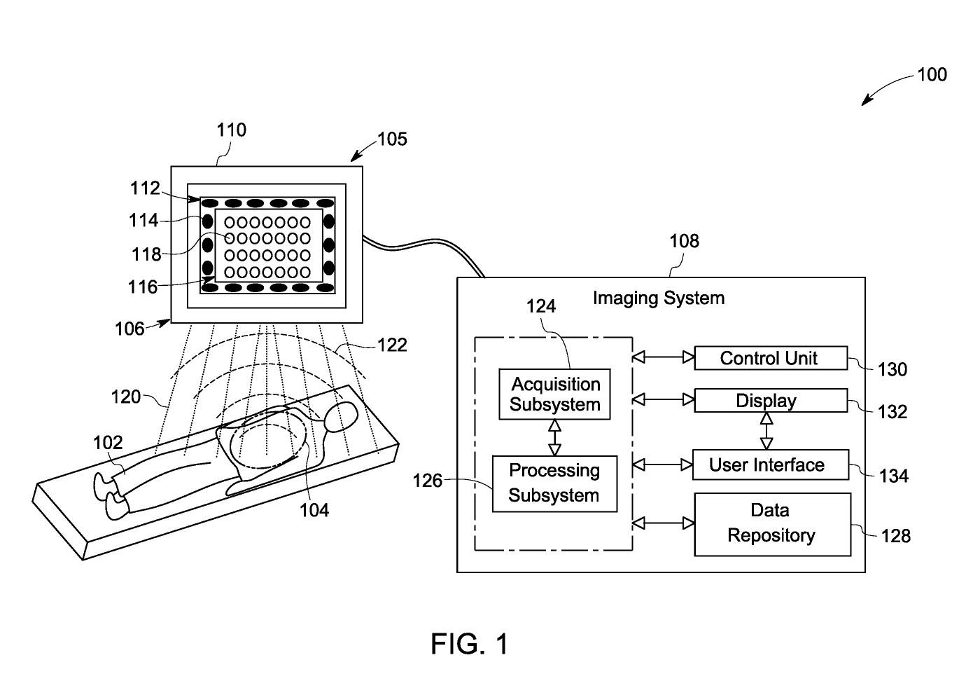

FIG. 1 is a block diagram of an exemplary system 100 for use in diagnostic imaging, in accordance with aspects of the present specification. As will be appreciated by one skilled in the art, the figures are for illustrative purposes and are not drawn to scale. In one example, the system 100 may be a photoacoustic imaging system. The system 100 is configured to aid a clinician such as a radiologist in imaging a subject of interest to determine presence of a diseased state, monitor efficacy of treatment, and the like. More particularly, the system 100 may be configured to facilitate imaging of a region of interest in the subject of interest and enhancing clinical workflow by providing a low cost, handheld, portable imaging system 100.

In one embodiment, the system 100 may be configured to acquire image data from a subject of interest such as a patient 102. In particular, the system 100 may be configured to acquire image data corresponding to a region of interest 104 in the patient 102, in one example. Moreover, in one example, a tissue in the region of interest 104 may be imaged. The system 100 may also include an image data acquisition device 105 that is operatively coupled to a medical imaging system 108 and configured to acquire the image data. In one embodiment, the image acquisition device 105 may include a probe, where the probe may include an invasive probe, or a non-invasive or external probe that is configured to aid in the acquisition of image data. In accordance with aspects of the present specification, the image data acquisition device 105 may include an exemplary photoacoustic transducer assembly 106 configured to irradiate the tissue being imaged and detect acoustic waves/signals generated by the tissue in response to the delivered irradiation. It may be noted that the terms photoacoustic transducer assembly and photoacoustic ultrasound transducer assembly may be used interchangeably.

In certain other embodiments, the image data may also be acquired via one or more sensors (not shown) that may be disposed on the patient 102. By way of example, the sensors may include physiological sensors (not shown) such as electrocardiogram (ECG) sensors and/or positional sensors such as electromagnetic field sensors or inertial sensors. These sensors may be operationally coupled to a data acquisition device, such as an imaging system, via leads (not shown), for example. Use of one or more detectors or detector arrays for acquiring image data is also envisaged.

The photoacoustic imaging system 100 may also include a medical imaging system 108. In one embodiment, the medical imaging system 108 may be in operative association with the image data acquisition device 105. Alternatively, the image data acquisition device 105 may be an integral part of the medical imaging system 108. Also, in the present example, the medical imaging system 108 may be an ultrasound imaging system.

It should be noted that although the exemplary embodiments illustrated hereinafter are described in the context of a medical imaging system, other imaging systems and applications such as industrial imaging systems and non-destructive evaluation and inspection systems, such as pipeline inspection systems, liquid reactor inspection systems, are also contemplated. Additionally, the exemplary embodiments illustrated and described hereinafter may find application in multi-modality imaging systems that employ the photoacoustic imaging system in conjunction with other imaging modalities, position-tracking systems or other sensor systems. For example, the multi-modality imaging system may include a photoacoustic imaging system-positron emission tomography (PET) imaging system. Furthermore, it should be noted that although the exemplary embodiments illustrated hereinafter are described in the context of a medical imaging system, such as an ultrasound imaging system, use of other imaging systems, such as, but not limited to, a computed tomography (CT) imaging system, a contrast enhanced ultrasound imaging system, an X-ray imaging system, an optical imaging system, a positron emission tomography (PET) imaging system, a magnetic resonance imaging (MRI) system and other imaging systems is also contemplated in accordance with aspects of the present specification.

In a presently contemplated configuration, the medical imaging system 108 may be an ultrasound imaging system. Moreover, the medical imaging system 108 may include an acquisition subsystem 124 and a processing subsystem 126, in one embodiment. The acquisition subsystem 124 of the medical imaging system 108 may be configured to acquire image data representative of one or more anatomical regions of interest 104 in the patient 102 via the image data acquisition device 105, in one embodiment.

As previously noted, the currently available photoacoustic imaging systems typically use a laser, such as a YAG laser as a source of irradiation. However, use of these lasers increases the footprint and cost of the imaging systems and inhibits the portability of these imaging systems. Moreover, the currently available photoacoustic imaging systems provide a single point laser source and operate at a very low repetition rate, thereby increasing the scan time. The shortcomings of the currently available photoacoustic imaging systems may be circumvented via use of an exemplary photoacoustic transducer assembly 106. In particular, the exemplary system 100 obviates the need for an expensive and bulky laser source, thereby minimizing the cost and footprint of the photoacoustic imaging system 100.

In one embodiment, the image data acquisition device 105 may include an exemplary the photoacoustic transducer assembly 106. Furthermore, the photoacoustic transducer assembly 106 may include a first portion 112 and a second portion 116 disposed on a substrate 110. The first portion 112 of the photoacoustic transducer assembly 106 may be representative of a source configured to irradiate the tissue in the region of interest 104. Also, the second portion 116 of the photoacoustic transducer assembly 106 may be representative of a detector configured to detect signals generated by the tissue in response to the irradiation delivered by the source. Although the image data acquisition device 105 and more particularly the photoacoustic transducer assembly 106 of FIG. 1 is shown as having a footprint that irradiates a substantially large portion of the patient 102, it may be noted that the photoacoustic transducer assembly 106 may have a footprint that is configured to cover or encompass a relatively smaller portion in or about the region of interest 104 of the patient 102. By way of example, the photoacoustic transducer assembly 106 may be sized such that the photoacoustic transducer assembly 106 is configured to encompass the tissue being imaged in the region of interest 104 in the patient 102.

Furthermore, in a presently contemplated configuration, the source 112 may include a plurality of source elements 114 arranged in a determined pattern on one or more sides of the substrate 110. In one example, the plurality of source elements 114 may be arranged along a periphery of one side of the substrate 110. Each source element 114 may include a laser source, in certain embodiments. In one embodiment, each source element 114 or laser source may include a laser diode. Moreover, in other embodiments, each source element 114 or laser source may include a fiber laser. Additionally, in accordance with further aspects of the present specification, the plurality of source elements 114 or laser sources may include a combination of laser diodes and fiber lasers.

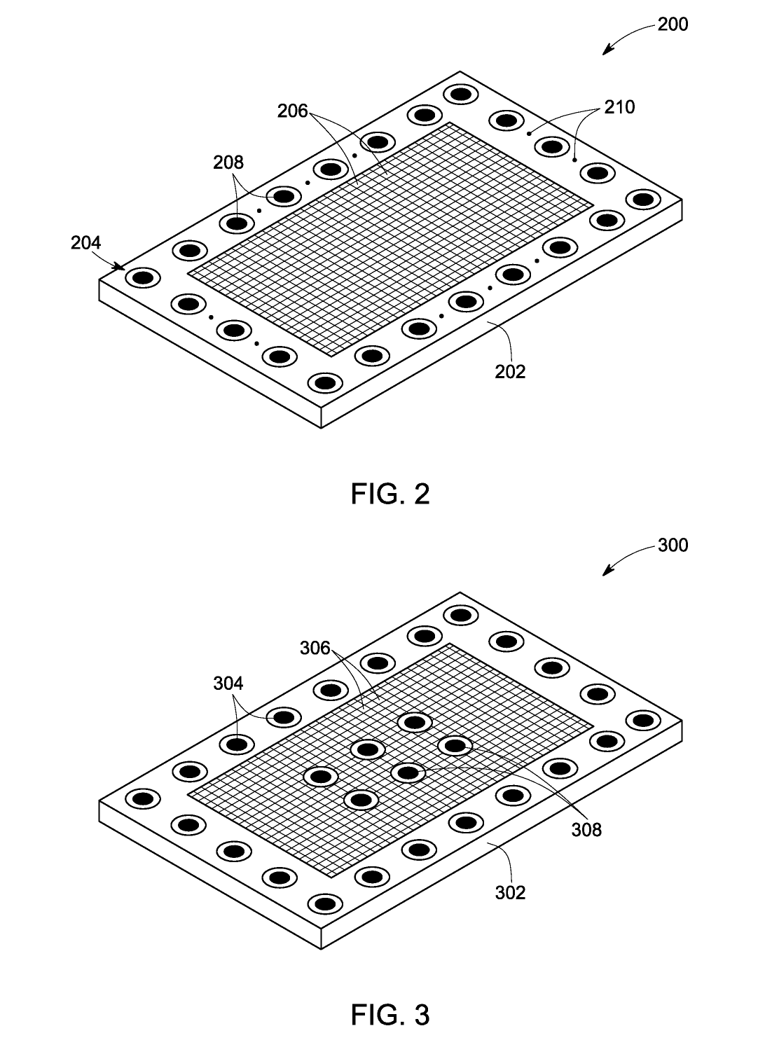

In a similar fashion, the detector 116 may include a plurality of detector elements 118 arranged in a determined pattern on the one or more sides of the substrate 110. More particularly, in one embodiment, the plurality of detector elements 118 may be disposed in a region of the substrate 110 that is away from the periphery of the substrate 110. By way of example, in a presently contemplated configuration, the plurality of detector elements 118 may be disposed in the center of the substrate 110. Specifically, the plurality of detector elements 118 may be disposed on the substrate 110 such that the plurality of source elements 114 surrounds the plurality of detector elements 118. The plurality of detector elements 118 may include ultrasound transducers, such as, but not limited to, capacitive micromachined ultrasonic transducers (cMUTs), in one embodiment. The exemplary photoacoustic transducer assembly 106 will be described in greater detail with reference to FIGS. 2-5.

Additionally, the plurality of source elements 114 may be configured to deliver irradiating energy to the tissue in the region of interest 104 being imaged. Reference numeral 120 may be generally representative of the irradiation or irradiating energy delivered to the tissue being imaged by the plurality of source elements 114. In one example, the region of interest 104 may be irradiated by non-ionizing laser pulses from the plurality of source elements 114. In another example, radio-frequency pulses may be employed to irradiate the tissue in the region of interest 104. Subsequent to the irradiation energy being delivered to the tissue by the plurality of source elements 114, the tissue in the region of interest 104 may absorb at least a portion of the delivered energy 120. The rate of absorption is dependent upon the type of tissue in the region of interest 104. Furthermore, the energy absorbed by the tissue may be converted into heat. This heat may lead to an increase in temperature of the tissue, which in turn may result in a transient thermal/thermoelastic expansion of the tissue. As a consequence of the thermal/thermoelastic expansion of the tissue, the tissue being irradiated may generate one or more signals in response to the irradiation 120. In one example, the one or more signals may include acoustic waves 122. In one example, the acoustic waves 122 may include ultrasonic emission. Moreover, the acoustic/ultrasonic waves 122 so generated may be detected by plurality of detector elements 118. The acoustic waves 122 detected by the plurality of detector elements 118 may be communicated to the medical imaging system 108 for further processing.

In one example, the medical imaging system 108 may include an acquisition subsystem 124 and a processing subsystem 126. The acquisition subsystem 124 may be configured to acquire image data corresponding to the region of interest 104 in the patient 102 via the image data acquisition device 105. The acquired image data may include the acoustic waves 122 detected by the plurality of detector elements 118. In addition, the processing subsystem 126 may be configured to process the acquired image data to generate one or more images representative of the region of interest 104. The image data acquired by the acquisition subsystem 124 and/or the images generated by the processing subsystem 126 in the medical imaging system 108 may be employed to aid a clinician in diagnosing a condition of the patient 102.

Furthermore, in certain embodiments, the processing subsystem 126 may also be coupled to a storage system, such as the data repository 128, where the data repository 128 may be configured to store the processed image data. The image data acquired by the acquisition subsystem 124 may also be stored in the data repository 128 (see FIG. 1). In certain embodiments, the data repository 128 may include a local database. Alternatively, the data repository 128 may include an archival site, a database, or an optical data storage article. In certain embodiments, the acquisition subsystem 124 may be configured to acquire images stored in the optical data storage article. It may be noted that the optical data storage article may be an optical storage medium, such as a compact disc (CD), a digital versatile disc (DVD), multi-layer structures, such as DVD-5 or DVD-9, multi-sided structures, such as DVD-10 or DVD-18, a high definition digital versatile disc (HD-DVD), a Blu-ray disc, a near field optical storage disc, a holographic storage medium, or another like volumetric optical storage medium, such as, for example, two-photon or multi-photon absorption storage format.

Additionally, the medical imaging system 108 may include a control unit 130 configured to control an operation of the photoacoustic transducer assembly 106. In particular, the control unit 130 may be configured control the operation of the plurality of source elements 114 and/or the operation of the plurality of detector elements 118. It may be noted that a desired power of the irradiation to be delivered to the tissue may be dependent on the tissue being imaged or an application. By way of example, if a superficial tissue is being imaged, a lower power of irradiation may suffice. However, if a deeper tissue is being imaged, then a higher power of irradiation may be desired. Accordingly, it may be desirable to energize one or more source elements 114 in the plurality of source elements 114 to ensure delivery of the desired power of irradiation, thereby enhancing the quality of imaging.

In accordance with aspects of the present specification, the control unit 130 may be configured to selectively energize and/or de-energize one or more source elements 114. To that end, in one example, the control unit 130 may be configured to determine a desired power of irradiation to be delivered to the tissue being imaged. The control unit 130 may be configured to energize and/or de-energize one or more source elements 114 based on the determined desired power. Implementing the control unit 130 as described hereinabove aids in ensuring that adequate power of irradiation is delivered to the tissue being imaged.

Further, as illustrated in FIG. 1, the medical imaging system 108 may include a display 132 and a user interface 134. In certain embodiments, such as in a touch screen, the display 132 and the user interface 134 may overlap. Also, in some embodiments, the display 132 and the user interface 134 may include a common area. In accordance with aspects of the present specification, the display 132 of the medical imaging system 108 may be configured to display one or more images corresponding to the region of interest 104, an indicator of any diagnosis generated by the medical imaging system 108 based on the acquired image data, and the like.

In addition, the user interface 134 of the medical imaging system 108 may include a human interface device (not shown) configured to aid the clinician in manipulating image data displayed on the display 132. The human interface device may include a mouse-type device, a trackball, a joystick, a stylus, or a touch screen configured to aid the clinician in identifying the one or more regions of interest requiring therapy. However, as will be appreciated, other human interface devices, such as, but not limited to, a touch screen, may also be employed. Furthermore, in accordance with aspects of the present specification, the user interface 134 may be configured to aid the clinician in navigating through the images generated by the medical imaging system 108. Additionally, the user interface 134 may also be configured to aid in manipulating and/or organizing the displayed images displayed on the display 132.

Turning now to FIG. 2, one exemplary embodiment 200 of a photoacoustic transducer assembly, such as the photoacoustic transducer assembly 106 of FIG. 1, in accordance with aspects of the present specification, is depicted. In a presently contemplated configuration, the photoacoustic transducer assembly 200 may include a substrate 202. The substrate 202 may be formed using glass, silicon, quartz, flexible plastic, and the like. In one embodiment, the substrate 202 may have a thickness in a range from about 100 μm to about 1 mm. Moreover, in one embodiment, the substrate 202 may have a rectangular shape. However, in other embodiments, the substrate 202 may have other shapes, such as, but not limited to, a circular shape, a triangular shape, a square shape, and the like. Furthermore, the substrate 202 may be a flexible substrate, in one embodiment. Alternatively, the substrate 202 may be a non-flexible substrate.

In accordance with further aspects of the present specification, the photoacoustic transducer assembly 200 may include a plurality of source elements 204. Additionally, in one example, the plurality of source elements 204 may be arranged on one side/surface of the substrate 202. In one embodiment, the plurality of source elements 204 may include laser sources. In one embodiment, the laser sources may include laser diodes 208, fiber lasers 210, or a combination thereof. Furthermore, the plurality of source elements 204 may be arranged in a determined pattern on one or more sides of the substrate 202. In one example, the determined pattern may include an array of the source elements 204. Moreover, in a presently contemplated configuration, the plurality of source elements 204 may be arranged along a periphery of the substrate 202. In one embodiment, the plurality of source elements 204 may include substantially similar laser sources such as the laser diodes 208 or the fiber lasers 210. More particularly, the plurality of source elements 204 may include laser sources having substantially similar wavelengths. In alternative embodiments, the plurality of source elements 204 in the form of laser sources having different wavelengths may be used. The plurality of source elements 204 may be configured to irradiate a tissue in a region of interest being imaged. In one example, the irradiating energy delivered by the plurality of source elements 204 may include a pulsed laser excitation.

Although the example of FIG. 2 depicts the plurality of source elements 204 as being arranged in a single row along the periphery of the substrate 202, in other embodiments, the source elements 204 may be arranged in a plurality of rows and/or columns along the periphery of the substrate 202. In this example, source elements 204 in one row and/or column may include laser diodes or fiber lasers having a first wavelength, while source elements 204 in another row/column may include source elements 204 having a second wavelength, where the second wavelength is different from the first wavelength. Moreover, the source elements 204 may have a size in a range from about 0.5 mm to about 2.5 mm, in one example.

With continuing reference to FIG. 2, the photoacoustic transducer assembly 200 may also include a plurality of detector elements 206. The plurality of detector elements 206 may be arranged in a determined pattern, such as an array. Moreover, in one embodiment, the plurality of detector elements 206 may be disposed on the substrate 202 such that the plurality of detector elements 206 is surrounded by the plurality of source elements 204. In one example, the plurality of detector elements 206 may include cMUTs. Furthermore, the plurality of detector elements 206 may be configured to sense or detect acoustic waves generated by the tissue being imaged in response to the pulsed laser excitation delivered by the plurality of source elements 204.

FIG. 3 illustrates another exemplary embodiment 300 of a photoacoustic transducer assembly for use in the photoacoustic imaging system 100 of FIG. 1, in accordance with aspects of the present specification. The example of the photoacoustic transducer assembly 300 depicted in FIG. 3 is substantially similar to the photoacoustic transducer assembly 200 of FIG. 2. In accordance with further aspects of the present specification, in addition to the source elements disposed along the periphery of the substrate, one or more source elements may also be interspersed amongst the plurality of detector elements, as depicted in FIG. 3.

In a presently contemplated configuration, the photoacoustic transducer assembly 300 may include a substrate 302. The substrate 202 may be formed using glass, silicon, quartz, and the like. Also, in one embodiment, the substrate 302 may have a square shape, a circular shape, a triangular shape, a rectangular shape, and the like. Furthermore, the substrate 302 may be a flexible substrate or a non-flexible substrate.

Moreover, in the example of FIG. 3, the photoacoustic transducer assembly 300 may include a first plurality of source elements 304 arranged along a periphery of the substrate 302. Furthermore, the first plurality of source elements 304 may include substantially similar laser sources such as laser diodes or fiber lasers, where the laser sources may be configured to irradiate a tissue in a region of interest being imaged. In one embodiment, the irradiating energy delivered by the plurality of source elements 304 may include a pulsed laser excitation.

Additionally, the photoacoustic transducer assembly 300 may also include a plurality of detector elements 306 arranged in a determined pattern. More particularly, in one embodiment, the plurality of detector elements 306 may be disposed on the substrate 302 such that the plurality of detector elements 306 is surrounded by the first plurality of source elements 304.

In accordance with other aspects of the present specification, in addition to the first plurality of source elements 304 disposed along the periphery of the substrate 302, the photoacoustic transducer assembly 300 may also include a second plurality of source elements 308. In one embodiment, the second plurality of source elements may be disposed at a location other than the periphery of the substrate 302. By way of example, the second plurality of source elements 308 may be interspersed amongst the plurality of detector elements 306.

Implementing the photoacoustic transducer assembly as described hereinabove provides a photoacoustic transducer assembly having an ultrasound sensor array in the form of the plurality of detector elements arranged as described hereinabove. Furthermore, the plurality of source elements is arranged along the periphery of the substrate such that the plurality of source elements surrounds the plurality of detector elements. The tissue being imaged may be irradiated by the plurality of source elements that surrounds the plurality of detector elements. Additionally, use of low cost laser diodes and/or fiber lasers as the plurality of source elements aids in circumventing the high costs associated with use of a YAG laser in the presently available systems.

Furthermore, while the YAG laser used in the currently available systems adds to the bulk of the system, use of the laser diodes and/or fiber lasers in lieu of the bulky laser system advantageously reduces the footprint of the exemplary system 100, thereby facilitating a low cost, handheld, portable design of the photoacoustic imaging system 100. In addition, the dimensions of the image data acquisition device and more particularly the dimensions of the exemplary photoacoustic transducer assembly may be customized based on the size and/or type of the tissue being imaged. In one example, the photoacoustic transducer assembly may have a size in a range from about 1 cm to about 5 cm. Moreover, while the currently available systems that include the YAG laser provide a single source of irradiation, the plurality of source elements used in the exemplary photoacoustic transducer assembly provides multiple sources of irradiation, thereby allowing a larger coverage of the tissue being imaged at a given point in time, and consequently reducing the scan time. Additionally, the photoacoustic imaging system 100 permits use of laser diodes and/or fiber lasers having different wavelengths, thereby allowing use of the system 100 in spectroscopy applications.

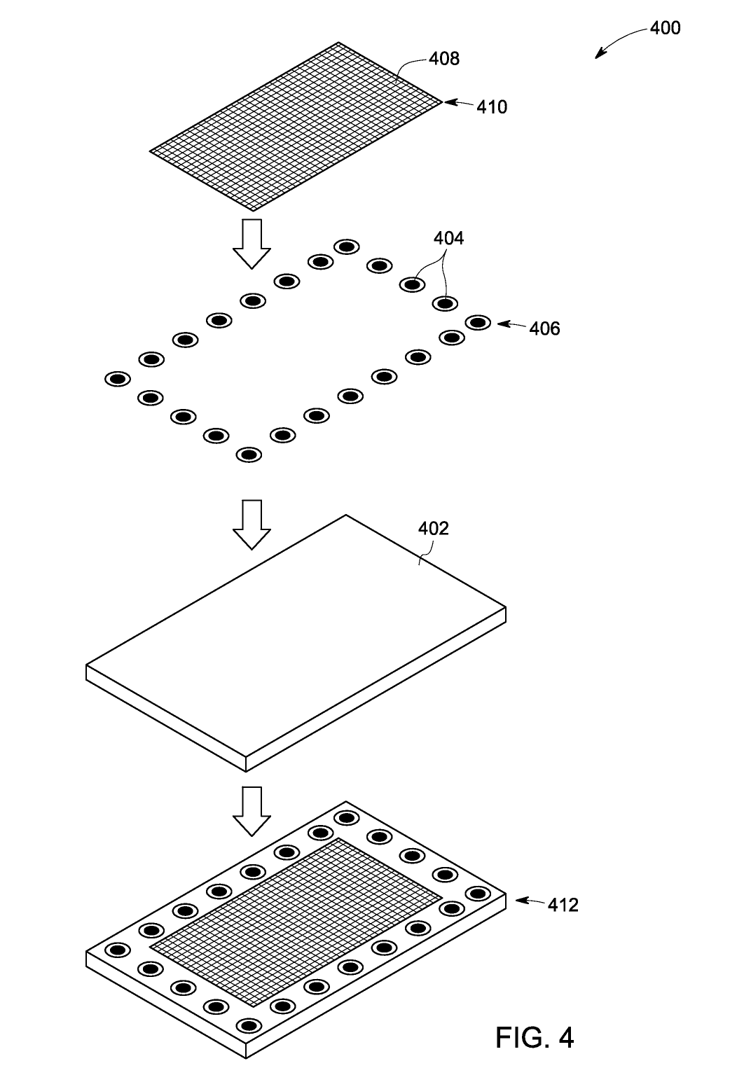

Referring now to FIG. 4, a diagrammatical representation 400 of a method of making an exemplary photoacoustic transducer assembly such as the photoacoustic transducer assembly 200 of FIG. 2, in accordance with aspects of the present specification, is depicted. The method 300 is described with reference to the elements of FIGS. 2-3.

A substrate having a first side and a second side, such as a substrate 402 may be provided. The substrate may be formed using glass, silicon, quartz, flexible plastic, and the like. Also, as previously noted, the substrate 402 may have a thickness in a range from about 100 μm to about 1 mm. Furthermore, the substrate 402 may have a rectangular shape. However, use of other shapes for the substrate 402 such as, but not limited to, a circular shape, a triangular shape, a square shape, and the like, is also envisaged. In one embodiment, the substrate 402 may be a non-flexible substrate, while in other embodiments, the substrate 402 may be a flexible substrate. It may also be noted that in one embodiment, the substrate 402 may be sized such that a photoacoustic transducer assembly may be configured to encompass the tissue being imaged in the region of interest in a patient such as the patient 102 of FIG. 1.

Subsequently, a plurality of source elements 404 may be disposed on one or more sides of the substrate 402. As previously noted, the plurality of source elements may include laser diodes or fiber lasers. In one embodiment, the plurality of source elements may be arranged in a determined pattern, such as an array 406. Moreover, the array 406 of the plurality of source elements 404 may be disposed on the first side of the substrate 402. In a presently contemplated configuration, the array 406 of the plurality of source elements 404 may include an arrangement of the plurality of source elements 404 such that the plurality of source elements 404 is arranged along a periphery of the substrate 402.

Additionally, in one embodiment, the plurality of source elements 404 may include substantially similar laser sources such as laser diodes and/or fiber lasers. In one example, the plurality of source elements 404 may include laser sources having substantially similar wavelengths. In certain other embodiments, the plurality of source elements 404 may include laser sources having different wavelengths. Moreover, the array 406 of the plurality of source elements 404 of FIG. 4 is depicted as including the plurality of source elements 404 arranged in a single row along the periphery of the substrate 402. However, in certain other embodiments, the source elements 404 may be arranged in a plurality of rows and/or columns along the periphery of the substrate 402. In this example, all the source elements 404 may include laser diodes and/or fiber lasers having substantially similar wavelengths. However, in another embodiment, the plurality of source elements 404 may include laser diodes and/or fiber lasers having different wavelengths. By way of example, the source elements 404 in one row and/or column may include laser diodes and/or fiber lasers having a first wavelength, while source elements 404 in another row/column may include laser diodes and/or fiber lasers having a second wavelength, where the second wavelength is different from the first wavelength. As previously noted, the plurality of source elements 404 may be configured to irradiate a tissue in a region of interest being imaged. In one embodiment, the irradiating energy delivered by the plurality of source elements 404 may include a pulsed laser excitation.

Furthermore, the plurality of source elements 404 may be operatively coupled to the substrate 402. In one example, techniques such as, but not limited to, bonding, soldering, gluing, and the like may be used to operatively couple the source elements 404 to the substrate 402. Moreover, in another example, one or more through-hole vias may be provided in the substrate 402. The laser sources in the form of the laser diodes and/or fiber lasers may be arranged in these through-hole vias to operatively couple the laser sources to the substrate 402.

Moreover, a plurality of detector elements 408 may be arranged in a determined pattern to form an array 410 of detector elements 408. The array 410 of the plurality of detector elements 408 may be disposed on the substrate 402 such that the plurality of detector elements 408 is surrounded by the plurality of source elements 404. In one example, the plurality of detector elements 408 may include cMUTs. It may be noted that the plurality of detector elements 408 may be configured to sense or detect acoustic waves generated by the tissue being imaged in response to the pulsed laser excitation delivered by the plurality of source elements 406. In addition, the plurality of detector elements 408 may be operatively coupled to the substrate 402. In one example, techniques such as, but not limited to, bonding, gluing, soldering and the like may be used to operatively couple the detector elements 408 to the substrate 402. As previously noted with FIG. 3, in certain embodiments, in addition to the source elements 404 disposed along the periphery of the substrate 402, one or more source elements may also be interspersed amongst the plurality of detector elements 408. An exemplary photoacoustic transducer assembly 412 may be formed as depicted in FIG. 4.

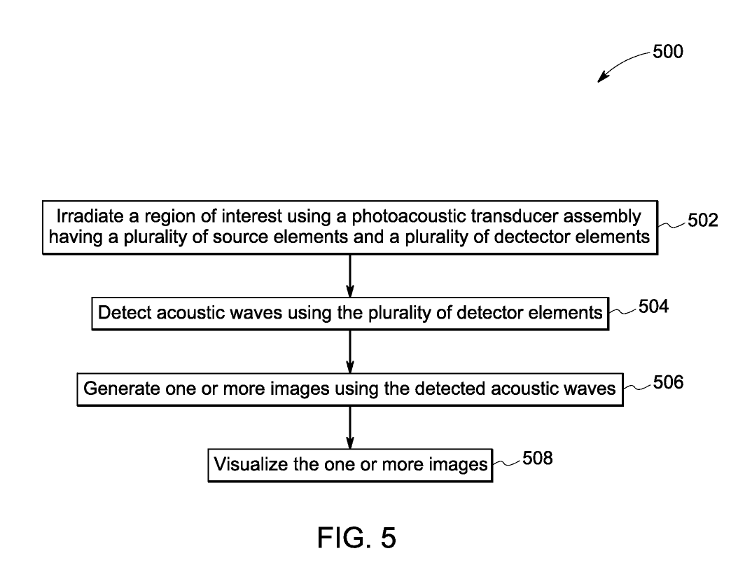

Turning now to FIG. 5, a flow chart of exemplary logic 500 for a method for imaging using an exemplary photoacoustic transducer assembly such as the exemplary photoacoustic transducer assembly 200 of FIG. 2 is depicted. It may be noted that the method of FIG. 5 is described in terms of the various components of FIGS. 1-4.

As previously noted, the exemplary photoacoustic transducer assembly 200 may include the plurality of source elements 204 arranged along the periphery on a first side of the substrate 202. Also, the plurality of source elements 204 may include laser diodes and/or fiber lasers. Additionally, the photoacoustic transducer assembly 200 may also include the plurality of detector elements 206 disposed on the first side of the substrate 202 such that the plurality of detector elements 206 is surrounded by the plurality of source elements 204. The plurality of detector elements 206 may include cMUTs, as previously noted.

During an imaging session, a probe, such as the image data acquisition device 105 of FIG. 1 may be positioned on or about the patient being imaged such that the photo acoustic transducer assembly 200 is disposed on or about the tissue being imaged in the patient 102.

It may be desirable to acquire image data representative of the tissue being imaged. Accordingly, at step 502, the region of interest and more particularly the tissue being imaged may be irradiated using the exemplary photoacoustic transducer assembly 200. In one embodiment, one or more source elements in the plurality of source elements may be selectively energized to facilitate irradiating the tissue being imaged. As previously noted, the plurality of source elements in the form of laser diodes and/or fiber lasers may be configured to deliver pulsed laser excitation to the tissue being imaged.

Additionally, based on a desired energy level or power to be delivered to the tissue, the plurality of source elements may be selectively energized. In one embodiment, the control unit 130 of FIG. 1 may be employed to facilitate the selective energizing of the plurality of source elements. It may be noted that the desired energy may be determined based on the type of tissue being imaged. By way of example, if a superficial tissue is being imaged, then a lower energy level may be sufficient to image the superficial tissue. However, a higher level of energy may be desirable to image deeper tissues. The control unit 130 may be configured to accordingly selectively energize a desired number of source elements to provide the desired levels of pulsed laser excitation.

As previously noted, a portion of the delivered energy may be absorbed by the tissue being imaged and converted into heat. The heat so generated in the tissue may result in a transient thermoelastic expansion, which in turn may cause generation of acoustic waves. The generated acoustic/ultrasonic waves may be detected by the plurality of detector elements, as indicated by step 504.

The detected acoustic waves may be communicated to an imaging system, such as the ultrasound imaging system 108 of FIG. 1. The imaging system 108 may be configured to receive the detected acoustic waves and generate one or more images representative of the tissue being imaged, as depicted by step 506. Subsequently, as indicated by step 508, these images may be visualized on a display, such as the display 132 of FIG. 1. A clinician, such as an ultrasound technician or a radiologist may then use these images to diagnose a disease state in the patient, if any.

The method 500 may be described in a general context of computer executable instructions. Generally, computer executable instructions may include routines, programs, objects, components, data structures, procedures, modules, functions, and the like that perform particular functions or implement particular abstract data types. In certain embodiments, the computer executable instructions may be located in computer storage media, such as a memory, local to the imaging system 108 (see FIG. 1) and in operative association with a processing subsystem. In certain other embodiments, the computer executable instructions may be located in computer storage media, such as memory storage devices, that are removed from the imaging system. Moreover, the method imaging includes a sequence of operations that may be implemented in hardware, software, or combinations thereof.

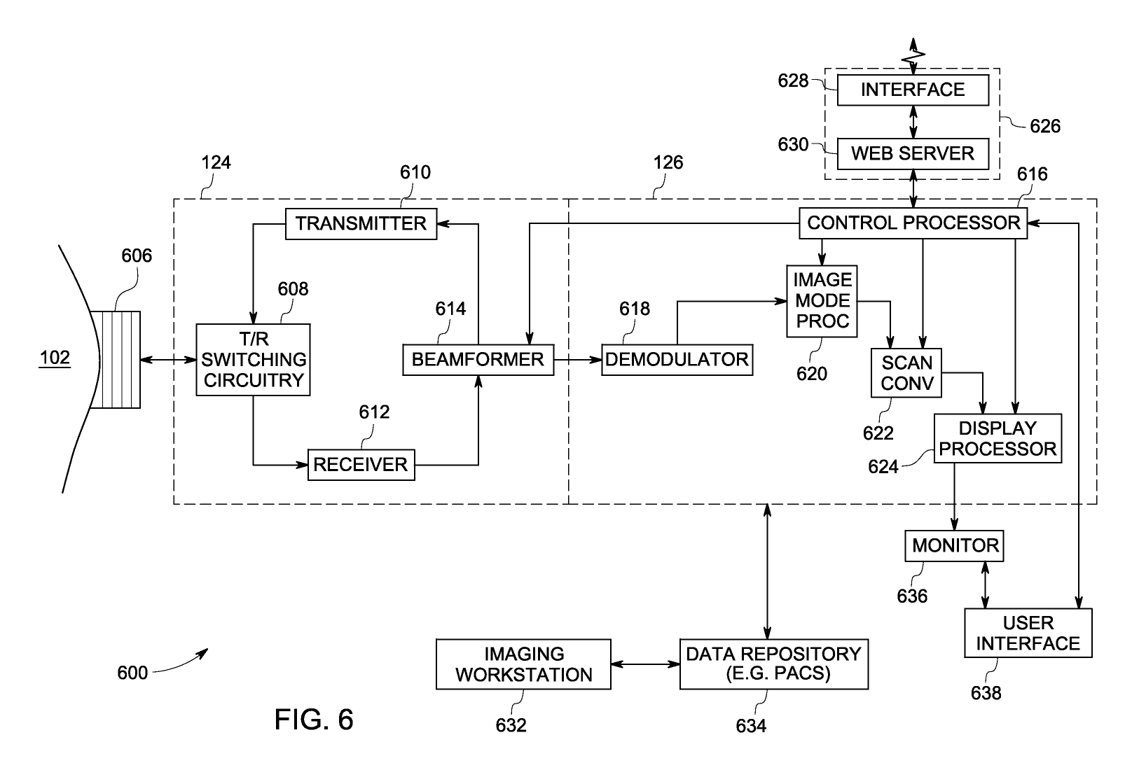

As previously noted with reference to FIG. 1, the medical imaging system 108 may include an ultrasound imaging system. FIG. 6 is a block diagram of an embodiment of an ultrasound imaging system 600 depicted in FIG. 1. The ultrasound system 600 includes an acquisition subsystem, such as the acquisition subsystem 124 of FIG. 1 and a processing subsystem, such as the processing subsystem 126 of FIG. 1. In one embodiment, the acquisition subsystem 124 may include a transducer assembly 606. Furthermore, in one embodiment, the transducer assembly 606 may include the exemplary photoacoustic transducer assembly 126 of FIG. 1. However, in certain embodiments, the transducer assembly 606 may be external to the imaging system 600 and more particularly to the acquisition subsystem 124. In this example, the transducer assembly 606 may be operatively coupled to the acquisition subsystem 124.

In addition, the acquisition subsystem 124 may include transmit/receive switching circuitry 608, a transmitter 610, a receiver 612, and a beamformer 614. It may be noted that in certain embodiments, the transducer assembly 606 may be disposed in an image data acquisition device such as the image data acquisition device 105 (see FIG. 1). Also, in certain embodiments, the transducer assembly 606 may include a plurality of source elements such as the source elements 204 of FIG. 2 and a plurality of detector elements such as the detector elements 206 of FIG. 2 (not shown) arranged in a spaced relationship to form the transducer assembly.

The processing subsystem 126 may include a control processor 616, a demodulator 618, an imaging mode processor 620, a scan converter 622 and a display processor 624. The display processor 624 may be further coupled to a display monitor 636, such as the display 132 (see FIG. 1), for displaying images. User interface 638, such as the user interface area 134 (see FIG. 1), may interact with the control processor 616 and the display monitor 636. The control processor 616 may also be coupled to a remote connectivity subsystem 626 including a remote connectivity interface 628 and a web server 630. The processing subsystem 126 may be further coupled to a data repository 634, such as the data repository 128 of FIG. 1, configured to receive and/or store image data. The data repository 634 may be configured to interact with an imaging workstation 632.

The aforementioned components may be dedicated hardware elements such as circuit boards with digital signal processors or may be software running on a general-purpose computer or processor such as a commercial, off-the-shelf personal computer (PC). The various components may be combined or separated according to various embodiments of the invention. Thus, those skilled in the art will appreciate that the present ultrasound imaging system 600 is provided by way of example, and the present specifications are in no way limited by the specific system configuration.

In the acquisition subsystem 124, the transducer assembly 606 may be disposed such that the transducer assembly 606 is in contact with the patient 102 (see FIG. 1). The transducer assembly 606 is coupled to the transmit/receive (T/R) switching circuitry 608. Also, the T/R switching circuitry 608 is in operative association with an output of transmitter 610 and an input of the receiver 612. The output of the receiver 612 is an input to the beamformer 614. In addition, the beamformer 614 is further coupled to the input of the transmitter 610 and to the input of the demodulator 618. The beamformer 614 is also operatively coupled to the control processor 616 as shown in FIG. 6.

In the processing subsystem 126, the output of demodulator 618 is in operative association with an input of the imaging mode processor 620. Additionally, the control processor 616 interfaces with the imaging mode processor 620, the scan converter 622 and the display processor 624. An output of imaging mode processor 620 is coupled to an input of scan converter 622. Also, an output of the scan converter 622 is operatively coupled to an input of the display processor 624. The output of display processor 624 is coupled to the monitor 636.

The ultrasound system 600 transmits ultrasound energy into the subject such as the patient 102 and receives and processes backscattered ultrasound signals from the subject 102 to create and display an image. To generate a transmitted beam of ultrasound energy, the control processor 616 sends command data to the beamformer 614 to generate transmit parameters to create a beam of a desired shape originating from a certain point at the surface of the transducer assembly 606 at a desired steering angle. The transmit parameters are sent from the beamformer 614 to the transmitter 610. The transmitter 610 uses the transmit parameters to properly encode transmit signals to be sent to the transducer assembly 606 through the T/R switching circuitry 608. The transmit signals are set at certain levels and phases with respect to each other and are provided to individual transducer elements such as the source elements of the transducer assembly 606. The transmit signals excite the transducer elements to emit irradiating energy or waves with the same phase and level relationships. As a result, a transmitted beam of irradiating energy is formed in the patient 102 within a scan plane along a scan line when the transducer assembly 606 is acoustically coupled to the patient 102 by using, for example, ultrasound gel. The process is known as electronic scanning.

The transducer assembly 606 may be a two-way transducer. When the irradiating energy is transmitted into the patient 102, the tissue being imaged may absorb at least a portion of the delivered irradiating energy. The absorbed energy may result in a thermoelastic expansion of the tissue, which in turn results in the generation of acoustic or ultrasound waves. The acoustic or ultrasound waves may be detected by the detector elements in the transducer assembly 606. The transducer assembly 606 and more particularly, the detector elements in the transducer assembly 606 may be configured to receive the acoustic waves at different times, depending on the distance into the tissue they return from and the angle with respect to the surface of the transducer assembly 606 at which they return. The detector elements may be configured to convert the ultrasound energy from the acoustic waves into electrical signals.

The electrical signals are then routed through the T/R switching circuitry 608 to the receiver 612. The receiver 612 amplifies and digitizes the received signals and provides other functions such as gain compensation. The digitized received signals corresponding to the backscattered waves received by each transducer element at various times preserve the amplitude and phase information of the backscattered waves.

The digitized signals are sent to the beamformer 614. The control processor 616 sends command data to beamformer 614. The beamformer 614 uses the command data to form a receive beam originating from a point on the surface of the transducer assembly 606 at a steering angle typically corresponding to the point and steering angle of the previous irradiating energy transmitted along a scan line. The beamformer 614 operates on the appropriate received signals by performing time delaying and focusing, according to the instructions of the command data from the control processor 616, to create received beam signals corresponding to sample volumes along a scan line in the scan plane within the patient 102. The phase, amplitude, and timing information of the received signals from the various transducer elements may be used to create the received beam signals.

The received beam signals may be communicated to the processing subsystem 126. The demodulator 618 demodulates the received beam signals to create pairs of I and Q demodulated data values corresponding to sample volumes within the scan plane. Demodulation is accomplished by comparing the phase and amplitude of the received beam signals to a reference frequency. The I and Q demodulated data values preserve the phase and amplitude information of the received signals.

The demodulated data is transferred to the imaging mode processor 620. The imaging mode processor 620 uses parameter estimation techniques to generate imaging parameter values from the demodulated data in scan sequence format. The imaging parameters may include parameters corresponding to various possible imaging modes such as B-mode, color velocity mode, spectral Doppler mode, and tissue velocity imaging mode, for example. The imaging parameter values are passed to the scan converter 622. The scan converter 622 processes the parameter data by performing a translation from scan sequence format to display format. The translation includes performing interpolation operations on the parameter data to create display pixel data in the display format.

The scan converted pixel data is sent to the display processor 624 to perform any final spatial or temporal filtering of the scan converted pixel data, to apply grayscale or color to the scan converted pixel data, and to convert the digital pixel data to analog data for display on the monitor 636. The user interface 638 is coupled to the control processor 616 to allow a user to interface with the ultrasound system 600 based on the data displayed on the monitor 636.

Furthermore, the foregoing examples, demonstrations, and process steps such as those that may be performed by the system may be implemented by suitable code on a processor-based system, such as a general-purpose or special-purpose computer. It should also be noted that different implementations of the present specification may perform some or all of the steps described herein in different orders or substantially concurrently, that is, in parallel. Furthermore, the functions may be implemented in a variety of programming languages, including but not limited to C++ or Java. Such code may be stored or adapted for storage on one or more tangible, machine readable media, such as on data repository chips, local or remote hard disks, optical disks (that is, CDs or DVDs), memory or other media, which may be accessed by a processor-based system to execute the stored code. Note that the tangible media may include paper or another suitable medium upon which the instructions are printed. For instance, the instructions may be electronically captured via optical scanning of the paper or other medium, then compiled, interpreted or otherwise processed in a suitable manner if necessary, and then stored in the data repository or memory.

The various systems and methods for photoacoustic imaging described hereinabove provide a framework for a low cost imaging system. Moreover, the systems and methods presented herein automatically provide desired levels of irradiation to the tissue being imaged, which in turn enhances the quality of imaging, while minimizing net scan time for a clinician, and thereby improving clinical workflow.

Use of the compact laser diodes and/or fiber lasers in lieu of the bulky YAG laser reduces the footprint of the photoacoustic imaging system, thereby allowing a portable, handheld design of the photoacoustic imaging system. Moreover, use of the low cost laser diodes and/or fiber lasers substantially reduces the cost of the photoacoustic imaging system. In addition, use of laser diodes and/or fiber lasers having different wavelengths allows use of the photoacoustic imaging system in spectroscopy applications.

Furthermore, use of different arrangements of multiple laser diodes provides enhanced irradiation to the tissue as opposed to the use of a single light source in the presently available systems. In addition, use of multiple laser diodes also provides a boost to the level of the irradiating signals.

While only certain features of the disclosure have been illustrated and described herein, many modifications and changes will occur to those skilled in the art. It is, therefore, to be understood that the appended claims are intended to cover all such modifications and changes as fall within the true spirit of the disclosure.

1. A photoacoustic transducer assembly for imaging a subject of interest, comprising:
a substrate;
a first plurality of source elements disposed on one or more sides of the substrate, wherein the first plurality of source elements is arranged along a periphery of the one or more sides of the substrate and configured to irradiate a region of interest in the subject of interest; and
a plurality of detector elements disposed on the one or more sides of the substrate, wherein the plurality of detector elements is surrounded by the first plurality of source elements and configured to detect one or more signals generated by the region of interest in response to the irradiation.

2. The photoacoustic transducer assembly of claim 1, wherein a size of the photoacoustic transducer assembly may be customized based on the region of interest being imaged.

3. The photoacoustic transducer assembly of claim 1, wherein the each of source elements in the first plurality of source elements comprises a laser source.

4. The photoacoustic transducer assembly of claim 1, wherein the first plurality of source elements comprises one or more laser diodes, one or more fiber lasers, or combinations thereof.

5. The photoacoustic transducer assembly of claim 4, wherein the one or more laser diodes comprise laser diodes having substantially similar wavelengths, and wherein the one or more fiber lasers comprise fiber lasers having substantially similar wavelengths.

6. The photoacoustic transducer assembly of claim 4, wherein the one or more laser diodes comprise laser diodes having different wavelengths, and wherein the one or more fiber lasers comprise fiber lasers having different wavelengths.

7. The photoacoustic transducer assembly of claim 1, wherein each of the plurality of detector elements comprises a capacitive micromachined ultrasound transducer.

8. The photoacoustic transducer assembly of claim 1, wherein the irradiation delivered by the plurality of source elements to the region of interest comprises a pulsed laser excitation, radio-frequency pulses, or a combination thereof.

9. The photoacoustic transducer assembly of claim 1, wherein the one or more signals detected by the plurality of detector elements comprise ultrasound signals.

10. The photoacoustic transducer assembly of claim 1, further comprising a second plurality of source elements disposed at a location other than the periphery of the substrate.

11. A photoacoustic imaging system for imaging a subject of interest, comprising:
an image data acquisition device configured to irradiate a region of interest in the subject of interest, wherein the image data acquisition device comprises a photoacoustic transducer assembly, and wherein the photoacoustic transducer assembly comprises:
a substrate;
a first plurality of source elements disposed on one or more sides of the substrate, wherein the first plurality of source elements is arranged along a periphery of the one or more sides of the substrate and configured to irradiate the region of interest in the subject of interest;
a plurality of detector elements disposed on the one or more sides of the substrate, wherein the plurality of detector elements is surrounded by the first plurality of source elements and configured to detect one or more signals generated by the region of interest in response to the irradiation;
an imaging device operatively coupled to the image data acquisition device and comprising:
an acquisition subsystem configured to acquire image data corresponding to the subject of interest from the image data acquisition device; and
a processing subsystem in operative association with the acquisition subsystem and configured to process the acquired image data to generate one or more images corresponding to the region of interest in the subject of interest.

12. The photoacoustic imaging system of claim 11, wherein the imaging device is an ultrasound imaging system.

13. The photoacoustic imaging system of claim 11, further comprising a display configured to visualize the one or more images corresponding to the region of interest in the subject of interest.

14. The photo acoustic imaging system of claim 11, further comprising a control unit configured to control operation of the plurality of source elements, the plurality of detector elements, or both the plurality of source elements and the plurality of detector elements.

15. The photoacoustic imaging system of claim 14, wherein the control unit is operatively coupled to the image data acquisition device, the imaging device, or both the image data acquisition device and the imaging device.

16. The photoacoustic imaging system of claim 14, wherein the control unit is configured to:
determine a desired level of energy of the irradiation to be delivered to the region of interest; and
selectively energize one or more source elements in the first plurality of source elements based on the desired level of energy of irradiation to be delivered to the region of interest.

17. The photoacoustic imaging system of claim 16, wherein the control unit is configured to vary the desired level of irradiation to be delivered to the region of interest based on a type of the region of interest.

18. The photoacoustic imaging system of claim 11, wherein the photoacoustic imaging system is a handheld system, a portable system, or a combination thereof.

19. A method for imaging a subject of interest, comprising:
irradiating a region of interest in the subject of interest using a photoacoustic transducer assembly, wherein the photoacoustic transducer assembly comprises:
a substrate;
a first plurality of source elements disposed on one or more sides of the substrate, wherein the first plurality of source elements is arranged along a periphery of the one or more sides of the substrate and configured to irradiate the region of interest in the subject of interest;
a plurality of detector elements disposed on the one or more sides of the substrate, wherein the plurality of detector elements is surrounded by the first plurality of source elements and configured to detect one or more signals generated by the region of interest in response to the irradiation;
detecting one or more signals generated by the region of interest in response to the irradiation; and
generating one or more images using the detected one or more signals.

20. The method of claim 19, wherein irradiating the region of interest in the subject of interest using the photoacoustic transducer assembly comprises irradiating the region of interest using the plurality of source elements having substantially similar wavelengths or different wavelengths.

21. The method of claim 19, further comprising visualizing the one or more images corresponding to the region of interest in the subject of interest on a display.

22. The method of claim 19, further comprising controlling an operation of the plurality of source elements, the plurality of detector elements, or both the plurality of source elements and the plurality of detector elements via a control unit.

23. The method of claim 22, wherein controlling operation of the plurality of source elements comprises:
determining a desired level of energy of the irradiation to be delivered to the region of interest; and
selectively energizing one or more source elements of the first plurality of source elements based on the desired level of energy of irradiation to be delivered to the region of interest.

24. The method of claim 23, further comprising varying the desired level of irradiation to be delivered to the region of interest based on a type of the region of interest.
